(19) United States
(12) Plant Patent Application Publication
(10) Pub. No.: US 2006/0041965 P1
(43) Pub. Date: Feb. 23, 2006

(54) White oak tree named AFT-O3

(50) QUERCUS ALBA; WHITE OAK

(76) Inventor: Walter F. Beineke, West LaFayette, IN (US)

Correspondence Address: BARNES & THORNBURG, LLP, P.O. BOX 2786, CHICAGO, IL, 60690-2786, US

(21) Appl. No.: 10/919,574

(22) Filed: Aug. 17, 2004

(51) Int. Cl. A01H 5/00

(52) U.S. Cl. PLT/225

(57) ABSTRACT

A new and distinct cultivar of white oak tree (Quercus alba L.) which is distinctly characterized by extremely rapid growth rate, strong central stem tendency, and excellent straightness, thereby producing excellent timber qualities, and annual acorn crops. This new variety of white oak tree was discovered by the applicant near Vallonia, Jackson County, Indiana in a front yard of a house. This selection has been designated as ‘AFTO-3’ in records maintained by the applicant on the performance of this selection and grafts made from the selection, and will be known hereafter as ‘AFTO-3’.

### LATIN NAME OF THE GENUS AND SPECIES

Quercus alba L.

### BACKGROUND OF THE INVENTION

This new variety of white oak tree (Quercus alba L.) was discovered by the applicant near Vallonia, Jackson County, Ind. in a front yard of a home. This selection has been designated as WO44 in records maintained by the applicant on the performance of this selection, and grafts made from the selection and will be known henceforth as AFT-O3. Both parents are unknown.

Before the house was constructed in the 1950's the area was a wooded pasture with many white oak and hickory trees present. This native stand was very open and the trees developed large, spreading crowns. The soil is almost pure sand and a part of a line of old sand dunes in the area. The soil is a Princeton sand and very droughty.

Therefore, it seems that this tree (the second largest in the area) was able to grow extremely fast in spite of the dry environment, indicating a superior genetic trait for rapid growth.

In two progeny test plantations consisting of a total of nearly 3,700 trees, seedlings from AFT-O3 have consistently outgrown 70 other seed sources thereby proving that this tree has superior genetic growth traits. Seedlings from AFT-O3 are extremely fast growing having ranked first (tied with AFT-O2, which is the subject of a separate patent application) out of the 70 families in the two progeny tests on diverse sites. The percent improvement in growth over the plantation average for the two tests was 19 and 18 percent. At age 10, AFT-O3 seedlings averaged 27.2 ft. tall at the first planting (plantation average was 22.9 ft) and therefore an improvement of 19% was obtained. At the second planting, AFT-03 seedlings averaged 11.2 ft. tall (plantation average was 9.5 ft.) and therefore an improvement of 18% was obtained.

### SUMMARY OF THE INVENTION

A new and distinct cultivar of white oak tree (Quercus alba L.) is distinctly characterized by extremely rapid growth rate, strong central stem tendency, and excellent straightness, thereby producing excellent timber qualities, the trait of commercial interest. AFT-O3 was approximately 105 years old when described at a location near Vallonia, Ind.

After the original clone was selected, and assigned an identity number of WO44 the aforesaid tree was reproduced by collecting scions from it and grafting these onto common white oak rootstocks at American Forestry Technologies, Inc., West Point, Ind. These asexual reproductions ran true to the originally discovered tree and to each other in all respects.

Color values used were from the Munsell Color Chart for Plant Tissues.

No disease or insect problems were detected.

AFT-O3 is hardy in USDA zones 5,6,7, and 8.

### DESCRIPTION OF THE DRAWINGS

FIG. 1 is a photograph showing the timber form of AFT-O3.

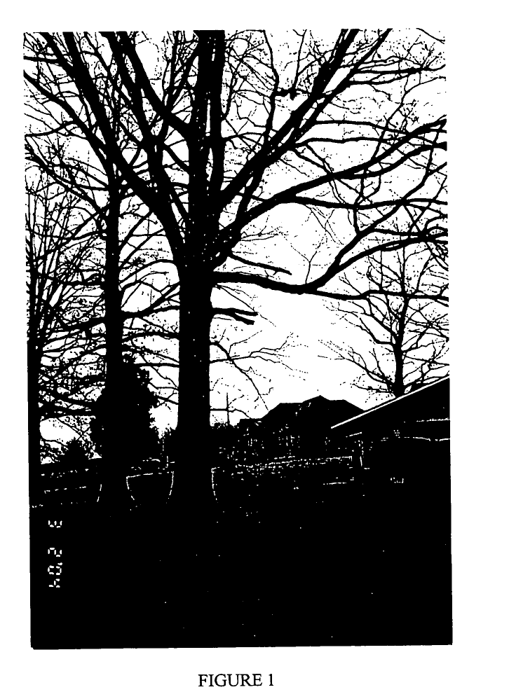

FIG. 2 is a photograph showing the leaves and nuts of AFT-O3.

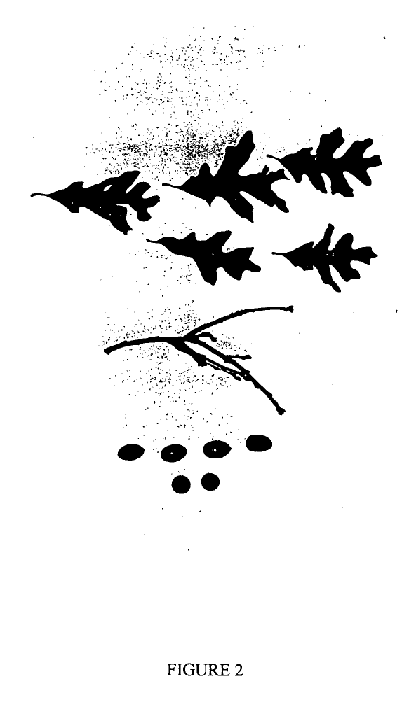

FIG. 3 is a photograph showing the bole of AFT-O3.

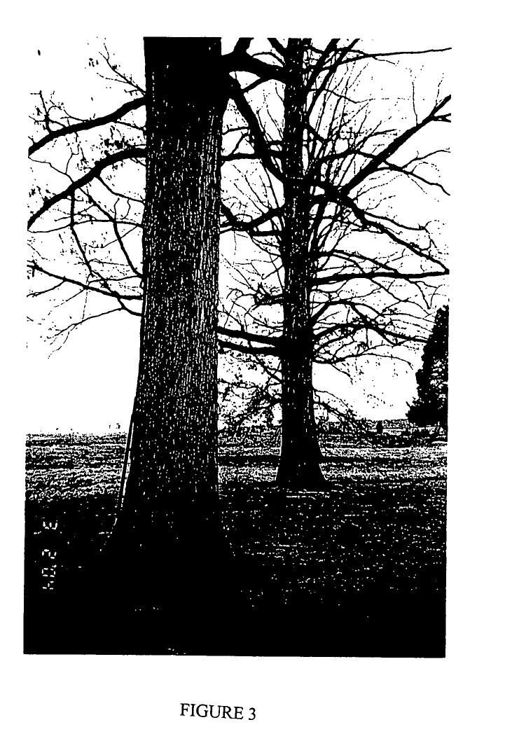

### BOTANICAL DESCRIPTION OF THE PLANTS

The botanical details of this new and distinct variety of white oak tree are as follows:
Tree:
   Size.—Large, 65 ft. tall; crown diameter of 60 ft.
   Vigor.—Vigorous.
   Growth rate.—Very rapid. Diameter at 4½ feet above the ground at 105 years was 35.9 inches for an average growth rate of 0.342 inches per year. For an old tree this is extremely rapid growth.
   Form.—Excellent timber form. AFT-O3 rates 1 on the 1 to 5 scale. Stem form was obtained by subjectively rating the straightness of the main stem on a scale of 1 to 5 with 1 representing a perfectly straight stem; 2, slight crook or deviation of the central stem (no crooks); 3, about average straightness; 4, several severe crooks or a single fork; and 5, a very crooked, forked and/or leaning central stem.
Branches and trunk:
   Branch and trunk color.—One year old branches, deep reddish — 10R3/4 on the Munsell Color Chart for Plant Tissues; Older branches, dark yellow-green — 7.5GY4/2 on the Munsell Color Chart for Plant Tissues; mature trunk and branches, mottled grays — 7.5YR5/2 and 2.5Y7/2 on the Munsell Color Chart for Plant Tissues.
   Branch and trunk characteristics.—One year old branches, slender, glabrous; older branches, smooth; mature trunk and branches, flaky, loose edges, ridged and furrowed, typical of the species.
   Internode length.—Average; 0.75 inches.
   Branch lenticels.—Light yellow-gray — 2.5Y8/2 on the Munsell Color Chart for Plant Tissues.

Leaves:
Leaves.—Size — Medium to small; average length including petiole — 5.1″; average width 2.78″.
Thickness.—Thick.
Texture.—Upper surface, smooth, glabrous; Lower surface, glabrous with white bloom.
Margins.—Rounded.
Color.—Upper Surface — dark green (2.5G4/6 by the Munsell Color Chart for Plant Tissues); Lower surface — light green (2.5G6/4 on the Munsell Color Chart for Plant Tissues).
Average distance between sinuses at leaf center.—0.78″.
Lobes.—Rounded, typical of species.
Average number of lobes.—7.8.
Petioles.—Length — 0.52″; Color-reddish (10R4/4 on the Munsell Color Chart for Plant Tissues).
Buds:
   Buds.—Typical of species — rounded, many imbricate scales, glabrous, smaller than average — 0.21″; Color — brownish red (5YR4/8 on the Munsell Color Chart for Plant Tissues).
Acorn:
   Nut shape.—Very long, elongated, oblong; length without cap — 1.1″, diameter 0.74 inches; Cap — width 0.71″, depth, very shallow — 0.27″.
   Flowering habit.—Flowers annually — unusual in the species. Flowers around May 15.
   Fruiting habit.—Produces annual and usually abundant acorn crops. This is very unusual in white oak which usually produces acorns only once every 3 to 5 years. Acorns ripen and fall on about October 20 — about average for most white oak.

### Documents Cited

O'Connor, Philip and Beineke, Walter F. (2004) “White Oak seedling performance: is seed source important?” Woodland Steward, (vol. 13, no. 1, pp. 10-11, 13 and 15).

I claim:

1. A new and distinct variety of white oak tree named ‘AFTO-3’ substantially as illustrated and described, which has extremely rapid growth rate, strong central stem tendency and excellent straightness, thereby producing excellent timber qualities, and annual acorn crops.
